(19) United States
(12) Patent Application Publication
(10) Pub. No.: US 2005/0069501 A1
(43) Pub. Date: Mar. 31, 2005

(54) Rapid temporary tooth whitening composition

(76) Inventors: Sayed Ibrahim, Somerset, NJ (US); Suman K. Chopra, Dayton, NJ (US); Michael Prencipe, West Windsor, NJ (US)

Correspondence Address: HARNESS, DICKEY, & PIERCE, P.L.C, 7700 BONHOMME, STE 400, ST. LOUIS, MO, 63105, US

(21) Appl. No.: 10/915,124

(22) Filed: Aug. 10, 2004

Related U.S. Application Data
(63) Continuation-in-part of application No. 10/641,963, filed on Aug. 15, 2003.

(51) Int. Cl. A61K 7/20

(52) U.S. Cl. 424/53

(57) ABSTRACT

A composition for the rapid, temporary whitening of teeth comprising an adhesive material and whitening particulates.

### CROSS-REFERENCE TO RELATED APPLICATIONS

This application is a continuation-in-part of U.S. patent application Ser. No. 10/641,963 filed on Aug. 15, 2003, the disclosure of which is incorporated herein by reference.

### INTRODUCTION

The present invention relates to rapid tooth whitening systems. Embodiments of the present invention include tooth whitening compositions comprising whitening particulates and adhesive agents.

Many substances such as tea and coffee that a person confronts or comes in contact with on a daily basis can “stain” or reduce the “whiteness” of one's teeth. Consumers consider clean, white teeth to be aesthetically desirable. Dull looking, stained teeth are objectionable to most people both on the basis of cosmetic appearance and also socially as an indication of poor oral hygiene. One whose teeth are white may enjoy more personal confidence and satisfaction and may even enjoy greater social acceptance.

Dental gel compositions containing active oxygen liberating ingredients such as hydrogen peroxide, urea peroxide, percarbonates and perborates of alkali and alkaline earth metals have been disclosed in the prior art for whitening teeth. Such gel compositions although effective for whitening teeth require multiple applications of the gel over a period of one to two weeks to provide the desired whitening effect and in some instances requires a skilled professional to apply the gel. However, it is in many situations it is desirable to cosmetically improve the appearance of the teeth, that is whiten the teeth rapidly and temporarily as in immediate anticipation of an important public appearance or social event.

There is a continuing need for a rapid temporary tooth whitening system which can be safely applied by the wearer and without the need for specially trained dental personnel. The tooth whitening to be achieved should be easily applied to the teeth and once applied to the teeth, be able to resist removal by mastication or solvating by foods and beverages for a period for at least 5 to 8 hours, but should be able to be easily removed by a wearer without harming the tooth. In this respect the prior art discloses tooth whitening coatings which can be applied to the teeth as a beauty aid providing immediate temporary whitening when applied to dental enamel in the same manner as one would apply a nail polish as for example as disclosed in U.S. Pat. Nos. 4,021,915; 4,032,627; 4,482,535 and 6,210,163, however there is a need for improved temporary whitening compositions that are effective, durable, and impart a “natural” look to the users teeth.

### SUMMARY

In one embodiment, the present invention provides a rapid tooth whitening composition for imparting a natural white appearance to dental enamel. The composition comprises an adhesive and a whitening particulate comprising hydroxyapatite.

In an embodiment of the present invention, a rapid tooth whitening composition for imparting a natural white appearance to dental enamel comprises a siloxane pressure sensitive adhesive and a whitening particulate.

In another embodiment, the present invention provides a method of imparting whiteness to a tooth in a mammal. The method comprises applying a composition to the tooth comprising a siloxane pressure sensitive adhesive and a whitening particulate.

It has been discovered that compositions and methods of this invention afford advantages over temporary whitening compositions among those known in the art including one or more of: enhanced whitening efficacy and aesthetics, improved adherence of the whitening composition to the tooth surface in the presence of saliva. Further uses, benefits and embodiments of the present invention are apparent from the description set forth herein.

### DESCRIPTION

The following definitions and non-limiting guidelines must be considered in reviewing the description of this invention set forth herein. The headings (such as “Introduction” and “Summary,”) and sub-headings (such as “Whitening Compositions” and “Methods”, for example) used herein are intended only for general organization of topics within the disclosure of the invention, and are not intended to limit the disclosure of the invention or any aspect thereof. In particular, subject matter disclosed in the “Introduction” may include aspects of technology within the scope of the invention, and may not constitute a recitation of prior art. Subject matter disclosed in the “Summary” is not an exhaustive or complete disclosure of the entire scope of the invention or any embodiments thereof. Classification or discussion of a material within a section of this specification as having a particular utility (e.g., as being an “active” or a “carrier” ingredient) is made for convenience, and no inference should be drawn that the material must necessarily or solely function in accordance with its classification herein when it is used in any given composition.

The citation of references herein does not constitute an admission that those references are prior art or have any relevance to the patentability of the invention disclosed herein. Any discussion of the content of references cited in the Introduction is intended merely to provide a general summary of assertions made by the authors of the references, and does not constitute an admission as to the accuracy of the content of such references. All references cited in the Description section of this specification are hereby incorporated by reference in their entirety.

The description and specific examples, while indicating embodiments of the invention, are intended for purposes of illustration only and are not intended to limit the scope of the invention. Moreover, recitation of multiple embodiments having stated features is not intended to exclude other embodiments having additional features, or other embodiments incorporating different combinations the stated of features. Specific Examples are provided for illustrative purposes of how to make and use the compositions and methods of this invention and, unless explicitly stated otherwise, are not intended to be a representation that given embodiments of this invention have, or have not, been made or tested.

As used herein, the words “preferred” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the word ‘include,” and its variants, is intended to be non-limiting, such that recitation of items in a list is not to the exclusion of other like items that may also be useful in the materials, compositions, devices, and methods of this invention.

As referred to herein, all compositional percentages are by weight of the total composition, unless otherwise specified.

### Tooth Whitening Compositions

In accordance with various embodiments of the present invention, a whitening composition is provided for imparting a natural white appearance to a tooth surface. The tooth surface is generally comprised of dental enamel, and the whitening composition contacts and preferably adheres to the dental enamel to impart an immediate discernable whitening effect, thus rapidly altering the color of the surface of the tooth. The present invention provides methods for whitening a tooth surface using compositions according to the present invention. As referred to herein, “tooth” or “teeth” refers to natural teeth, dentures, dental plates, fillings, caps, crowns, bridges, dental implants, and the like, and any other hard surfaced dental prosthesis either permanently or temporarily fixed within the oral cavity. As used herein, “whitening” refers to a change in visual appearance of a tooth, preferably such that the tooth has a brighter shade. Increase in whiteness of a dental surface can be observed visually, for example with the aid of color comparison charts or gauges, or measured by colorimetry, using any suitable instrument such as a Minolta Chromameter, e.g., model CR-400 (Minolta Corp., Ramsey, N.J.). The instrument can be programmed, for example, to measure Hunter Lab values or L*a*b* values according to the standard established by the International Committee of Illumination (CIE). The L*a*b* system provides a numerical representation of three-dimensional color space where L* represents a lightness axis, a* represents a red-green axis and b* represents a yellow-blue axis. The L* and b* axes are typically of greatest applicability to measurement of tooth whiteness. Increase in whiteness can be computed from differences in L*, a* and b* values before and after treatment, or between untreated and treated surfaces. A useful parameter is ΔE*, calculated as the square root of the sum of the squares of differences in L*, a* and b* values, using the formula:
ΔE*=[(ΔL*)²+(Δa*)²+(Δb*)²]^{1/2}
   A higher value of ΔE* indicates greater increase in whiteness. In various embodiments, the method of the present invention can effect a ΔE* of at least about 1, or at least about 3, or at least about 4, or at least about 5.

In one embodiment of the present invention, the whitening composition comprises an adhesive and a whitening particulate. The whitening composition can comprise additional ingredients, as recognized by one of skill in the art. The whitening composition can be incorporated into a dental strip or film form, or into a liquid product which can be applied to the surface of a tooth by manual application. In another embodiment, methods of whitening a surface of a tooth are provided.

### Adhesives

In various embodiments of the present invention, the whitening composition comprises an adhesive, which serves multiple functions, including enhancing adherence of the whitening composition to the surface of the tooth to be whitened. In such embodiments, the adhesive generally comprises one or more organic polymers or copolymers that are dispersed or dissolved in a volatile solvent.

A variety of organic polymers are useful as adhesives with the present invention. For example, one preferred class of adhesive polymers comprise siloxane polymers, which are also generally known in the art as “silicone” polymers. In certain embodiments of the present invention, the adhesive polymers in the composition are those in which a whitening particulate can be dispersed and are well known in the art. Many such silicone polymers are commercially available. In various embodiments, a preferred silicone-based adhesive polymer is a polyorganosiloxane. One such polyorganosiloxane is produced by condensing a silicone resin and an organosiloxane such as a polydiorganosiloxane. Such polymers are an elastomeric, tacky material, adhesion of which to dental enamel surfaces can be varied by altering the ratio of silicone resin to polydiorganosiloxane in the copolymer molecule. Preferably, the polymers are pressure sensitive polymers specifically designed for pharmaceutical use and are permeable to many drug compounds and find application for the transdermal application of various compounds. In one such embodiment, the silicone polymers are the copolymer product of mixing a silanol terminated polydiorganosiloxane such as polydimethyl siloxane with a silanol-containing silicone resin whereby the silanol groups of the polydiorganosiloxane undergo a condensation reaction with the silanol groups of the silicone resin so that the polydiorganosiloxane is lightly crosslinked by the silicone resin (that is, the polydiorganosiloxane chains are bonded together through the resin molecules to give chain branching and entanglement and/or a small amount of network character) to form the silicone polymers. A catalyst, for example, an alkaline material, such as ammonia, ammonium hydroxide or ammonium carbonate, can be mixed with the silanol-terminated polydiorganosiloxane and the silicone resin to promote this crosslinking reaction. By copolymerizing the silicone resin with the silanol terminated polydiorganosiloxane, there results a polymer with self adhering properties and the cohesive properties of a soft elastomer matrix characteristic of pressure sensitive polymers being distinguished from the hard, non-elastomeric properties of other silicone resins. In one embodiment, adhesive polymers used in the whitening composition are available from the Dow-Coming Company under the brand name BIO-PSA.

The modification of a ratio of silicone resin to polydiorganosiloxane modifies the tackiness of the hydrophilic polymer. This ratio can be in the range of about 70:30 to about 50:50. For example, the BIO-PSA silicone sold by Dow-Corning is available in three silicone resin to silicone polymer ratios namely, 65/35 (low tack), 60/40 (medium tack), 55/45 (high tack). The rapid tooth whitening composition in some embodiments according to the invention contains an organic adhesive dispersed or dissolved in the volatile solvent. For example, a polyorganosiloxane pressure sensitive adhesive is available dissolved in either ethyl acetate solvent or dimethicone.

In other embodiments of the present invention, useful polymers for adhesives include those such as Carbomers, such as carboxymethylene polymers including acrylic acid polymers and acrylic acid copolymers. Carboxypolymethylene is a slightly acidic vinyl polymer with active carboxyl groups. A carboxypolymethylene preferred for use in the practice of the present invention is a copolymer of acrylic acid cross linked with approximately 0.75% to approximately 1.5% polyallyl sucrose that is sold under the trade designation Carbopol 934, 974 by B.F. Goodrich. The adhesive is preferably a natural resin, particularly a resin selected from shellac resins, colophonium resins and modified colophonium resins. Another suitable group of adhesives is organic polymeric compositions represented by the group of alkyd resins, polyvinyl acetaldehydes, polyvinyl alcohols, polyvinyl acetates, poly(ethylene oxide), polyacrylates, ketone resins, polyvinylpyrrolidone, polyvinylpyrrolidone/vinyl acetate copolymer, polyethylene glycols of 200 to 1000 molecular weight and polyoxyethylene/polyoxopropylene block copolymers (Polyox), and silicone resins. As appreciated by one of skill in the art, the adhesive polymers may comprise copolymers or mixtures of polymers, including the adhesive polymers described herein.

In various embodiments, the adhesive polymer is present in the liquid whitening compositions of the present invention at a concentration of about 1 to about 80% by weight and preferably about 15 to about 40% by weight.

### Volatile Carrier

Certain embodiments of the present invention comprise carriers, especially those whitening compositions that are in liquid solution. Some suitable carriers for use in the preparation of the whitening compositions of the present invention include volatile solvents such water, ethanol and ethyl acetate and non-volatile, water soluble solvents such as triacetin. Generally the solvent in which the whitening particle is dispersed is present in the composition at a concentration of about 10 to about 80% by weight and preferably about 40 to about 70% by weight.

### Whitening Particulates

In various embodiments, the whitening composition comprises a whitening particulate. As used herein, the term “whitening particulate” can encompass a plurality of whiteness-imparting particulates or particles within the whitening composition. It should be noted that the plurality of particulates may comprise one or more different species of whitening particulate materials that are independently selected from one another. The selection of multiple whitening particles may be useful for modifying the overall color shade of the whitening composition. In various embodiments, the whitening particulates can be any white colored or white pigmented particles such as, for example, white mineral particles, white metal oxide particles, or a white polymer particles. As used herein, “white” is considered a color, and a “white” color can be any color commonly perceived as white, for example colors set forth in the Vita Shade Guide scale of whiteness, or colors that are perceived as whiter than those displayed in the Vita Shade Guide.

In various embodiments of the present invention, the average diameter of such particles can be from about 0.5 μm (microns) to about 500 microns, from about 10 microns to about 100 microns, or from about 20 microns to about 50 microns.

In some embodiments, white mineral particles can comprise a non-toxic mineral or salt that can impart a white color. In various embodiments, the whitening particulates can comprise a calcium phosphate. In various configurations, the calcium phosphate can have a structure selected from tetracalcium phosphate, amorphous calcium phosphate, alpha-tricalcium phosphate, beta-tricalcium phosphate and hydroxyapatite (Ca₅(OH)(PO₄)₃). The calcium phosphate in various embodiments, can be a substantially aqueous insoluble calcium phosphate and non-crystalline, poorly crystalline or crystalline form such as, for example, crystalline hydroxyapatite.

Hydroxyapatite has a similar physical structure as tooth enamel, and thus has a strong affinity to the tooth enamel surfaces, resulting in the hydroxyapatite particulates imparting a “natural” white appearance to the enamel surface, where they are used. The preparation of hydroxyapatite is well known to the art as disclosed in U.S. Pat. No. 4,274,879; U.S. Pat. No. 4,330,514; U.S. Pat. No. 4,324,772; U.S. Pat. No. 4,408,300; U.S. Pat. No. 4,097,935; U.S. Pat. No. 4,207,300. The hydroxyapatite whitening agent is present in the whitening composition of the present invention at a concentration of about 0.5 to about 60% by weight and preferably about 15 to about 40% by weight.

Non-limiting examples of a hydroxyapatite include Hydroxyapatite A1 (Himed, Old Bethpage, N.Y.). In some configurations, hydroxyapatite particles can comprise aggregates of smaller hydroxyapatite particles. In non-limiting example, such aggregates can have a mean diameter of from about 100 nm to about 1000 nm, and comprise hydroxyapatite particles having a mean diameter of from about 0.1 nm to about 10 nm.

The whitening particulates can be, in some embodiments, a metal oxide. The metal oxide can comprise any metal oxide that provides a white color, such as, for example, titanium oxide, aluminum oxide, tin oxide, calcium oxide, magnesium oxide, barium oxide, or a combination thereof.

The whitening particulates can be, in some embodiments, polymeric white-colored particles such as disclosed in U.S. Pat. No. 6,669,930 to Hoic. Polymeric white-colored particles can comprise, in non-limiting example, polyethylene (PE), polypropylene, ethylene/propylene copolymer, polytetrafluoroethylene (PTFE) or polyhexafluoropropene. In a non-limiting example, the polymeric white-colored particles can be polyethylene PE220, polypropylene, or PTFE as supplied by PreSperse, Inc., Somerset N.J. In a non-limiting example, the polymeric whitening particulates can comprise a polymer having a weight average molecular weight, a number average molecular weight, a Z-average molecular weight or a viscosity average molecular weight ranging from about 100 to about 10,000,000; from about 200 to about 5,000,000; from about 500 to about 1,000,000; from about 1,000 to about 500,000; from about 10,000 to about 100,000, or from about 20,000 to about 50,000.

In some embodiments, the whitening particulates can comprise pearlescent particles. In some configurations, the pearlescent particles can provide a white pearlescent appearance to a composition herein. “Pearlescence” and “pearlescent,” as used herein, refers to an optical property of a material in which the material can have a pearl-like, lustrous appearance. In some configurations, a pearlescent material can provide an appearance of depth. In some aspects, a pearlescent material can further provide an appearance of shine. Without being limited by theory, pearlescent particles are believed to partially reflect and partially refract incident light. The extent of partial refraction or reflection of incident light by a pearlescent material can depend on the angle of light incidence and/or the angle of viewing. Pearlescent particles used in the compositions described herein can provide aesthetic or cosmetic effects such as, for example, sparkle or luster.

Pearlescent particles can comprise a single mineral or chemical species, such as, for example a silicate such as mica, or bismuth oxychloride. By “mica” is meant any one of a group of hydrous aluminum silicate minerals with platy morphology and perfect basal (micaceous) cleavage. Mica can be, for example, sheet mica, scrap mica or flake mica, as exemplified by muscovite, biotite or phlogopite type micas. In some embodiments, the pearlescent particles can comprise a complex comprising more than one mineral or chemical species, such as, for example, mica coated with a metal oxide such as titanium oxide. Pearlescent particles can also be of biological origin, for example fish scale or mother-of-pearl. Certain pearlescent particles of biological origin can comprise calcium carbonate, such as, for example, pearl, mollusk shell such as mother-of-pearl obtained from oyster shell, or nacre.

In some embodiments, white pearlescent particles can be, for example, those described as Timiron® pigments, Biron® powders, Biron® dispersions or Nailsyn® dispersions (all registered trademarks of EM Industries, Inc. Hawthorne, N.Y., division of E. Merck). For example, mica titanium particles can be pearlescent particles such as Timiron® particles. White pearlescent mica titanium particles can be, for example “Silverwhite” Timiron® particles such as Timiron® Starluster MP-115, Timiron® Supersheen MP-1001, Timiron® Sparkle MP-47, Timiron® Supersilk MP-1005, Timiron® Pearl Flake MP-10, Timiron® Pearl Sheen MP-30, Timiron® Super Silver Fine, Timiron® Gleamer Flake MP-111, Timiron® Ultraluster MP-45, Timiron® Transwhite MP-18, Timiron® Diamond Cluster MP-149, Timiron® Super Silver, Timiron® Stardust MP-80, Timiron® Arctic Silver or Timiron® Snowflake MP-99.

A tooth-coating fluid of the present invention can comprise whitening particulates from about 0.01% (w/w) up to about 50% (w/w), from about 0.1% (w/w) up to about 20% (w/w), from about 1% (w/w) up to about 19% (w/w), from about 2% (w/w) up to about 18% (w/w), from about 3% (w/w) up to about 17% (w/w), from about 4% (w/w) up to about 16% (w/w), or from about 6% (w/w) up to about 15% (w/w). In some configurations, the whitening particulates (including pearlescent particles) can have an average size of from about 0.01 micron to about 500 microns, from about 0.5 micron to about 100 microns, or from about 2 microns to about 20 microns in diameter or longest dimension. Such particles as referenced herein are intended to include primary particles as well as aggregates of particles.

The plurality of whitening particulates can be independently comprised of a material selected from the group consisting of calcium phosphate compounds, titanium oxide, aluminum oxide, tin oxide, calcium oxide, magnesium oxide, polyethylene, polypropylene, ethylene/propylene copolymer, polytetrafluoroethylene, polyhexafluoropropene and combinations thereof. In one embodiment, the plurality of whitening particulates can comprise a calcium phosphate, such as a hydroxyapatite, in particular a crystalline hydroxyapatite.

### Additional Ingredients

In some embodiments, a sweetening material is employed in the tooth whitening composition of the present invention. Suitable sweetening agents include sodium saccharin, sodium cyclamate, xylitol, aspartame and the like, in concentrations of about 0.01 to about 1% by weight and preferably about 0.05 to about 0.75% by weight. Sodium saccharin is preferred for certain embodiments.

The tooth whitening compositions of the present invention optionally comprise a flavoring agent in various embodiments. Flavoring agents that are used in such embodiments include essential oils as well as various flavoring aldehydes, esters, alcohols, and similar materials. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Of these, the most commonly employed are the oils of peppermint, spearmint and wintergreen. The flavoring agent is incorporated in the whitening liquid composition of the present invention at a concentration of about 0.1 to about 2% by weight and preferably about 0.1 to about 0.5% by weight.

The compositions of the present invention optionally comprise an antimicrobial (e.g., antibacterial) agent. Any orally acceptable antimicrobial agent can be used, including Triclosan (5-chloro-2-(2,4-dichlorophenoxy)phenol); 8-hydroxyquinoline and salts thereof; zinc and stannous ion sources such as zinc citrate, zinc sulphate, zinc glycinate, sodium zinc citrate and stannous pyrophosphate; copper (II) compounds such as copper (II) chloride, fluoride, sulfate and hydroxide; phthalic acid and salts thereof such as magnesium monopotassium phthalate; sanguinarine; quaternary ammonium compounds, such as alkylpyridinium chlorides (e.g., cetylpyridinium chloride (CPC), combinations of CPC with zinc and/or enzymes, tetradecylpyridinium chloride, and N-tetradecyl-4-ethylpyridinium chloride,); bisguanides, such as chlorhexidine digluconate, hexetidine, octenidine, alexidine; halogenated bisphenolic compounds, such as 2,2′ methylenebis-(4-chloro-6-bromophenol); benzalkonium chloride; salicylanilide, domiphen bromide; iodine; sulfonamides; bisbiguanides; phenolics; piperidino derivatives such as delmopinol and octapinol; magnolia extract; grapeseed extract; thymol; eugenol; menthol; geraniol; carvacrol; citral; eucalyptol; catechol; 4-allylcatechol; hexyl resorcinol; methyl salicylate; antibiotics such as augmentin, amoxicillin, tetracycline, doxycycline, minocycline, metronidazole, neomycin, kanamycin and clindamycin; and mixtures thereof. A further illustrative list of useful antibacterial agents is provided in U.S. Pat. No. 5,776,435, Gaffar, et al., issued Jul. 7, 1998.

The compositions of the present invention optionally comprise an antioxidant. Any orally acceptable antioxidant can be used, including butylated hydroxyanisole (BHA), butylated hydroxytoluene (BHT), vitamin A, carotenoids, vitamin E, flavonoids, polyphenols, ascorbic acid, herbal antioxidants, chlorophyll, melatonin, and mixtures thereof.

The compositions of the present invention optionally comprise a saliva stimulating agent, useful for example in amelioration of dry mouth. Any orally acceptable saliva stimulating agent can be used, including without limitation food acids such as citric, lactic, malic, succinic, ascorbic, adipic, fumaric, and tartaric acids, and mixtures thereof. One or more saliva stimulating agents are optionally present in a saliva stimulating effective total amount.

The compositions of the present invention optionally comprise a breath freshening agent. Any orally acceptable breath freshening agent can be used, including without limitation zinc salts such as zinc gluconate, zinc citrate and zinc chlorite, α-ionone and mixtures thereof. One or more breath freshening agents are optionally present in a breath freshening effective total amount.

The compositions of the present invention optionally comprise an antiplaque (e.g., plaque disrupting) agent. Any orally acceptable antiplaque agent can be used, including without limitation stannous, copper, magnesium and strontium salts, dimethicone copolyols such as cetyl dimethicone copolyol, papain, glucoamylase, glucose oxidase, urea, calcium lactate, calcium glycerophosphate, strontium polyacrylates and mixtures thereof.

The compositions of the present invention optionally comprise an anti-inflammatory agent. Any orally acceptable anti-inflammatory agent can be used, including steroidal agents such as flucinolone and hydrocortisone, and nonsteroidal agents (NSAIDs) such as ketorolac, flurbiprofen, ibuprofen, naproxen, indomethacin, diclofenac, etodolac, indomethacin, sulindac, tolmetin, ketoprofen, fenoprofen, piroxicam, nabumetone, aspirin, diflunisal, meclofenamate, mefenamic acid, oxyphenbutazone, phenylbutazone, and mixtures thereof.

The compositions of the present invention optionally comprise an H2 antagonist. H2 antagonists useful herein include cimetidine, etintidine, ranitidine, ICIA-5165, tiotidine, ORF-17578, lupititidine, donetidine, famotidine, roxatidine, pifatidine, lamtidine, BL-6548, BMY-25271, zaltidine, nizatidine, mifentidine, BMY-52368, SKF-94482, BL-6341A, ICI-162846, ramixotidine, Wy-45727, SR-58042, BMY-25405, loxtidine, DA-4634, bisfentidine, sufotidine, ebrotidine, HE-30-256, D-16637, FRG-8813, FRG-8701, impromidine, L-643728, HB-408.4, and mixtures thereof.

The compositions of the present invention optionally comprise a desensitizing agent. Desensitizing agents useful herein include potassium citrate, potassium chloride, potassium tartrate, potassium bicarbonate, potassium oxalate, potassium nitrate, strontium salts, and mixtures thereof. Alternatively or in addition a local or systemic analgesic such as aspirin, codeine, acetaminophen, sodium salicylate or triethanolamine salicylate can be used.

The compositions of the present invention optionally comprise a nutrient. Suitable nutrients include vitamins, minerals, amino acids, and mixtures thereof. Vitamins include Vitamins C and D, thiamine, riboflavin, calcium pantothenate, niacin, folic acid, nicotinamide, pyridoxine, cyanocobalamin, para-aminobenzoic acid, bioflavonoids, and mixtures thereof. Nutritional supplements include amino acids (such as L-tryptophane, L-lysine, methionine, threonine, levocarnitine and L-carnitine), lipotropics (such as choline, inositol, betaine, and linoleic acid), fish oil (including components thereof such as omega-3 (N-3) polyunsaturated fatty acids, eicosapentaenoic acid and docosahexaenoic acid), coenzyme Q10, and mixtures thereof.

The compositions of the present invention optionally comprise biomolecules, such as bacteriocins, antibodies, and proteins. Suitable proteins include milk proteins and enzymes such as peroxide-producing enzymes, amylase, plaque-disrupting agents such as papain, glucoamylase, glucose oxidase, and “next generation” enzymes.

The compositions of the present invention optionally comprise additional ingredients, such as, plaque buffers such as urea, calcium lactate, calcium glycerophosphate and strontium polyacrylates; plant extracts; opacifying agents, and additional pigments and/or coloring agents.

The whitening composition of the present invention may be applied to tooth enamel by any suitable means, such as for example, in extruded form as a strip or as a liquid such as a paint-on liquid.

### Strip

The whitening composition of the present invention may be in the form of a layer of a strip prepared using a conventional solvent casting process. Strips among those useful herein comprise polymers, natural and synthetic woven materials, non-woven material, foil, paper, rubber and combinations thereof. Preferably the strip of material is substantially water insoluble. Suitable polymers include polyethylene, ethylvinylacetate, polyesters, ethylvinyl alcohol, fluoroplastics, and combinations thereof In various embodiments, the strip of material is generally less than about 1 mm (millimeter) thick, optionally less than about 0.05 mm thick, optionally about 0.001 to about 0.03 mm thick. The shape of the strip is any shape and size that covers the desired oral surface. In one embodiment, the length of the strip material is from about 2 cm (centimeter) to about 12 cm, in another embodiment from about 4 cm to about 9 cm. The width of the strip material will also depend on the oral surface area to be covered. The width of the strip is generally from about 0.5 cm to about 4 cm, in one embodiment from about 1 cm to about 2 cm. The strip material may comprise shallow pockets, optionally filled by a composition of this invention. Strips among those useful herein are disclosed in U.S. Pat. No. 6,514,484, Rajaiah et al. issued Feb. 4, 2003.

For example, in some embodiments, a strip is prepared by solvent casting, an adhesive polymer or resin such as colophonium and/or polyvinylpyrrolidone is dissolved in a sufficient amount of a compatible solvent such as ethanol. After a solution has been formed, the addition of whitening particulates in powder form (e.g., hydroxyapatite) follows and any other ingredients such as sweeteners or flavors. The solution is coated onto a suitable casting carrier material from which the formed strip can be easily released from without damage. The carrier material must have a surface tension which allows the solution to spread evenly across the intended carrier width without soaking in to form a destructive bond between the two substrates. Examples of suitable carrier materials include glass, stainless steel, teflon, polyethylene impregnated kraft paper. The strip may be dried to a solid usable form in a high temperature air bath using a drying oven, drying tunnel, vacuum drier, or any other suitable drying equipment at a temperature. Thereafter the strip formed on the carrier is peeled off the carrier surface and cut into pieces of suitable size and shape for consumer use and packed into a suitable container.

To use the whitening strip for the present invention, the strip when applied to the teeth surface will adhere to the teeth in an appropriate manner and within 1 to 60 minutes, the teeth surfaces will whiten to a natural appearance as the whitening particulates present in the strip matrix migrate to the tooth surfaces. In this regard, the whitening strip is formed to have a width dimension suitable to cover one or more teeth in a row (upper or lower). Therefore, the whitening strip may be applied to one or more of the upper set of teeth, or to one or more of the lower set of teeth either separately or simultaneously. The length dimension of the whitening strip is determined by the amount of coverage desired. In this regard, the number of teeth which it is desired to whiten will determine the dimensions for the whitening strip. For instance, it may be desired to only whiten the front teeth, which are most easily seen by others. Accordingly, the length of whitening strip can be reduced in this case, as compared to the case where it is desired to whiten all of the teeth. The duration of application of whitening strip to the teeth will depend upon the type and concentration of the whitening particulate, as well as the type and intensity of stain. The applied layer of whitening particulate will resist solvation removal by beverages or mastication for at least 2 to 8 hours and generally 3 to 5 hours. After the teeth are whitened to the satisfaction of the user, the portions of the strip can be easily removed by rinsing the mouth with water and brushing.

### Paint-On Tooth Whitener

In one preferred embodiment, the whitening composition is applied using a “paint-on” technique. A small application device, such as a brush or spatula is coated with a composition of this invention and the composition is then placed on a tooth surface. Preferably, the composition is spread evenly on such surfaces, in sufficient quantity to deliver whitening particulates to the stained surfaces.

The paint-on whitening composition of the present invention is prepared in the form of a flowable viscous liquid suspension containing the whitening particulate and is applied as such to the subject's teeth, by manual application, such as by painting the teeth with a soft applicator brush in the same manner as application of nail polish to a finger nail and without the intervention of a dentist or technological operations. Application by the user and evaporation or dissolution of the solvent leaves an adherent natural appearing white coating on the teeth which provides almost instantaneous whitening of the tooth enamel.

In various embodiments, the layer of tooth whitening composition applied to tooth enamel contains no ingredients imparting thereto an unacceptable taste or texture, rendering it unpleasant to the user while adhering strongly to tooth enamel. The composition is sufficiently adherent to tooth enamel to remain on the teeth for the applied whitening composition enabling the applied coating to resist the forces commonly applied by the lips and tongue as well a those forces encountered during normal mastication, as upon the evaporation or dissolution of the solvent in mouth after application a hard coating of whitening particulate forms in about 1 minute which coating is bonded securely to the tooth enamel to which it is applied. While the layer of applied paint-on whitening composition is in place, the user is to refrain from mastication. The whitening composition can be removed as and when required, at will, by an employment of standard oral hygiene procedures such as brushing or by rinsing with an alcoholic mouthwash.

### Methods

A method of imparting whiteness to a tooth in a mammal is provided in one embodiment of the present invention, where the method comprises applying a composition to the tooth. The composition comprises a siloxane pressure sensitive adhesive and a whitening particulate, such as those previously described. In certain embodiments, the siloxane pressure sensitive adhesive comprises a copolymer of polyorganosiloxane and silicone resin. Preferred whitening particulates are one or more materials selected from the group consisting of calcium phosphate, titanium oxide, aluminum oxide, tin oxide, calcium oxide, magnesium oxide, polyethylene, polypropylene, a copolymer of ethylene/propylene, polytetrafluoroethylene, polyhexafluoropropene and combinations thereof. Other preferred whitening particulates are selected from the group consisting of: tetracalcium phosphate, amorphous calcium phosphate, alpha-tricalcium phosphate, beta-tricalcium phosphate, hydroxyapatite and mixtures thereof. The whitening particulate preferably has an average diameter of from at least about 0.5 microns up to about 500 microns.

In an embodiment, the applying is achieved by contacting a film comprising the tooth whitening composition with a surface of a the tooth. In an alternate embodiment, the applying is achieved by contacting a liquid form of the tooth whitening composition with a surface of the tooth.

The whitening composition of the present invention is prepared by adding the ingredients of the composition in a suitable vessel such as a stainless steel tank provided with a mixer in one embodiment. In the preparation for the whitening composition, the ingredients are advantageously added to the mixer in the following order: solvent, adhesive materials, hydroxyapatite and any desired flavoring or sweetener. The ingredients are then mixed to form a homogeneous dispersion. When mixed and ready for extrusion or application directly as a paint-on whitener, the composition is a white colored opaque viscous liquid composition.

The present invention is illustrated by the following examples but is not to be limited thereby.

### EXAMPLE I

Formulations for whitening dental strips having the ingredients listed in Table I below.

TABLE I

| Strip Composition | A | B | C | D | E |
|---|---|---|---|---|---|
| Ethanol | 35.73 | 31.02 | 35.73 | 35.73 | 0 |
| Ethylacetate | 0 | 0 | 0 | 0 | 37.7 |
| Polyvinylpyrrolidone/ | 8.91 | 8.68 | 0 | 0 | 6.0 |
| vinyl acetate | | | | | |
| Polyvinylpyrrolidone | 0 | 0 | 8.91 | 0 | 2.9 |
| Hydroxyapatite | 19.62 | 24.32 | 19.62 | 19.62 | 19.6 |
| Colophonium | 35.74 | 31.02 | 35.74 | 35.74 | 31.7 |
| PEG 600 | 0 | 4.96 | 0 | 0 | 3.0 |
| Polyox | 0 | 0 | 0 | 7.0 | 0 |
| Water | 0 | 0 | 0 | 1.91 | 0 |
| Total (%) | 100.00 | 100.00 | 100.00 | 100.00 | 100.00 |

The strip compositions of Table I can be prepared by slowly adding the adhesive material (such as colophonium, polyvinyl pyrrolidone, polyvinyl pyrrolidone/vinyl acetate, Polyox, PEG 600 or mixtures thereof) into the solvent (ethanol, ethyl acetate) with vigorous stirring to form a translucent and viscous solution. Hydoxyapatite powder can be added to this solution and then thoroughly mixed. The resulting white mixture is cast onto a polyethylene coated kraft paper and should be allowed to dry. A white colored strip is formed which can be readily peeled off from the plate. The dry strip has an approximate thickness of about 0.1 to 0.25 mm.

### EXAMPLE II

A series of paint-on whitening compositions can be prepared to contain the ingredients listed in Table III below.

TABLE III
Liquid Paint-On Compositions

| | F | G | H | I | J | K |
|---|---|---|---|---|---|---|
| BIO-PSA* | 50 | 40 | 25 | 50 | 25 | 34.9 |
| Ethylacetate | 33 | 48 | 48 | 20 | 45 | 40 |
| Mineral Oil | 2 | 2 | 2 | — | 2 | — |
| Acetin | — | — | — | 10 | — | — |
| Hydroxyapatite | 5 | 10 | 25 | 20 | 25 | 25 |
| Carbopol 974 | — | — | — | — | 3 | 0.1 |

*silicone polymer based pressure sensitive adhesive

The compositions can be painted on tooth surfaces to impart a natural white appearance to the teeth. It is preferred that the paint-on composition remains on the tooth surface for about 3 to 8 hours before removing the coating.

1. A rapid tooth whitening composition for imparting a natural white appearance to dental enamel comprising an adhesive and a whitening particulate comprising hydroxyapatite.

2. A composition according to claim 1, wherein the composition contains from about 0.1 to about 99% by weight of the adhesive, and from about 0.5 to about 60% by weight of hydroxyapatite.

3. A composition according to claim 1, wherein said adhesive comprises a polyorganosiloxane polymer.

4. A composition according to claim 1, wherein said adhesive comprises a siloxane pressure sensitive adhesive.

5. A composition according to claim 1, wherein said adhesive comprises a silicone resin.

6. A composition according to claim 1, wherein said adhesive comprises a polymer selected from the group consisting of natural resins, modified natural resins and pressure sensitive adhesives.

7. A composition according to claim 1, wherein said adhesive comprises a polymer selected from the group consisting of polyvinylpyrrolidone, polyvinylpyrrolidone/vinyl acetate copolymers and polyethylene glycol.

8. A dental strip comprising a layer comprising the composition according to claim 1, wherein said layer is operable to adhere to a surface of a tooth.

9. A liquid tooth whitening composition comprising the composition according to claim 1, wherein the liquid tooth whitening composition adheres to a surface of a tooth.

10. A method for whitening the surface of a tooth comprising applying a composition according to claim 1 to a surface of the tooth.

11. A rapid tooth whitening composition for imparting a natural white appearance to dental enamel comprising a siloxane pressure sensitive adhesive and a whitening particulate.

12. A composition according to claim 11, wherein said whitening particulate is comprised of one or more materials selected from the group consisting of calcium phosphate, titanium oxide, aluminum oxide, tin oxide, calcium oxide, magnesium oxide, polyethylene, polypropylene, a copolymer of ethylene/propylene, polytetrafluoroethylene, polyhexafluoropropene and combinations thereof.

13. A composition according to claim 11, wherein said whitening particulate is selected from the group consisting of: tetracalcium phosphate, amorphous calcium phosphate, alpha-tricalcium phosphate, beta-tricalcium phosphate, hydroxyapatite and mixtures thereof.

14. A composition according to claim 11, wherein said siloxane pressure sensitive adhesive comprises a copolymer of polyorganosiloxane and silicone resin.

15. A composition according to claim 9, wherein said whitening particulate has an average diameter of from at least about 0.5 microns up to about 500 microns.

16. A method of imparting whiteness to a tooth in a mammal, the method comprising applying to the tooth a composition comprising a siloxane pressure sensitive adhesive and a whitening particulate.

17. A method according to claim 16, wherein said applying is achieved by contacting a film comprising said tooth whitening composition with a surface of a the tooth.

18. A method according to claim 16, wherein said applying is achieved by contacting a liquid form of said tooth whitening composition with a surface of the tooth.

19. A method according to claim 16, wherein said whitening particulate is comprised of one or more materials selected from the group consisting of calcium phosphate, titanium oxide, aluminum oxide, tin oxide, calcium oxide, magnesium oxide, polyethylene, polypropylene, a copolymer of ethylene/propylene, polytetrafluoroethylene, polyhexafluoropropene and combinations thereof.

20. A method according to claim 16, wherein said whitening particulate is selected from the group consisting of: tetracalcium phosphate, amorphous calcium phosphate, alpha-tricalcium phosphate, beta-tricalcium phosphate, hydroxyapatite and mixtures thereof.

21. A method according to claim 16, wherein said siloxane pressure sensitive adhesive comprises a copolymer of polyorganosiloxane and silicone resin.

22. A method according to claim 16, wherein said whitening particulate has an average diameter of from at least about 0.5 microns up to about 500 microns.
